(19) United States
(12) Patent Application Publication
(10) Pub. No.: US 2019/0198151 A1
(43) Pub. Date: Jun. 27, 2019

(54) Compression And/Or Contention Item, Pair Of Items, System And Method For Managing The Wearing Of An Item

(71) Applicant: Sigvaris AG, St. Gallen (CH)

(72) Inventors: Etienne GRENIER, Marcilly Le Chatel (FR); Coline MESMIN, Saint Romain Le Puy (FR)

(21) Appl. No.: 16/326,802

(22) PCT Filed: Aug. 31, 2017
(86) PCT No.: PCT/EP2017/071919

§ 371 Date: Feb. 20, 2019

(30) Foreign Application Priority Data
Sep. 1, 2016 (FR) 1658117

(51) Int. Cl. G16H 20/30

(52) U.S. Cl. CPC G16H 20/30; A41D 1/005

(57) ABSTRACT

A compression and/or contention item that is designed to be fitted to a limb of a wearer, in particular for treating venous insufficiency, the item incorporating a memory for storing data, which data can be read and in some cases written by a distinct electronic device, the memory including at least one datum for identifying the item, wherein the memory is configured to record a date on which the item is first worn, in an activation operation that is carried out on the day the item is first worn and that includes a transfer of data, including the date on which the item is first worn, from the electronic device to the memory. A system and a method for managing the wearing of an item is also contemplated.

### FIELD

The present invention concerns a compression and/or contention item designed to be fitted to a limb of a wearer, particularly for the treatment of venous insufficiency. The invention also concerns a pair of items. The invention also relates to a system and a method of managing the wearing of an item.

The field of the invention is that of compression and/or contention items, such as socks, stockings and sleeves, designed to be fitted to the upper or lower limbs of a person.

### BACKGROUND

In order to benefit from optimal therapeutic effect, it is essential that the wearer of the item comply with the recommendations of the manufacturer and the doctor. Different systems have therefore been developed to evaluate compliance, i.e. compliance with the therapeutic treatment. Some systems comprise labels for pairing items, in order to ensure that the wearer is equipped with a pair of items having characteristics suitable for his health condition. Other systems comprise surface sensors incorporated into the items, in order to detect their wearing.

For example, the documents DE102012012324, JP2014185411 and US2015145671 describe different systems for managing the wearing of items of clothing. In these examples, the items are provided with labels that can be read by a reader, particularly a mobile phone.

In DE102012012324, the system seeks to facilitate the pairing of items. Each item comprises an RFID label making it possible to identify that it belongs to a specific pair.

In JP2014185411, the system seeks to keep a record of items based on seasons, climate, function, etc.

In US2015145671, the system has numerous functionalities, but is relatively complex. According to a preferred embodiment, each item includes at least one surface sensor making it possible to detect the wearing thereof. The item also includes a clock enabling the data collected by the sensors to be dated. The date on which the item is first worn is recorded during the first detection by the sensor.

### SUMMARY

The purpose of the present invention is to propose a new solution for managing the wearing of a compression and/or contention item.

To that end, an object of the invention is a compression and/or contention item designed to be fitted to a limb of a wearer, particularly for the treatment of venous insufficiency, the item incorporating a memory for storing data, which can be read and for some written by a distinct electronic device provided for that purpose, the memory including at least one datum for identifying the item.

According to the invention, the memory is configured to record a date on which the item is first worn, in an activation operation that is carried out on the day the item is first worn and which comprises a transfer of data, including the date on which the item is first worn, from the electronic device to the memory.

Thus, the invention makes it possible to ensure that the date on which the item is first worn is known, which facilitates the monitoring of the treatment and makes it possible to warn the wearer when it is time to wear a new item, at the end of a predefined period of use.

According to other advantageous characteristics of the item according to the invention, taken alone or in combination:
The item is devoid of a sensor for detecting wearing by the wearer.
The item is devoid of a battery.
The memory includes at least one datum of identification of the items selected among the following: an item reference; a pair identifier, when two items must be worn simultaneously; an item type; and/or a date of manufacture.
The at least one datum of identification of the item is fixed and unchangeable in the memory.
The memory includes a wearer identifier, a site identifier and/or an owner identifier.
The item is equipped with RFID or NFC communication means for communication with the electronic device.
The communication means are activated only in the event data are read or written in memory by the electronic device. The rest of the time the item is “inert” in terms of emission of waves.

An object of the invention is also a pair composed of two items, such as those defined above. The activation operation is performed for each item of the pair, so that the said items have a common date on which they are first worn.

An object of the invention is also a system of managing the wearing of an item, comprising at least: one item as defined above; and an electronic device configured to read the data and write some data in the memory incorporated in the item.

According to other advantageous characteristics of the system according to the invention, taken in isolation or in combination:
The system comprises at least one pair of items.
The system comprises a remote server, in communication with the electronic device.
The system comprises additional electronic devices configured to read the data stored in the memory of the item.
A device identifier and a read time are recorded during an operation of reading the memory by one of the additional electronic devices.
The device identifier and the read time are recorded in the memory, in the electronic device and/or on the remote server.

The object of the invention is also a method for managing the wearing of an item as described above. The method comprises an activation operation performed on the day the item is first worn and which comprises a transfer of data, including the date on which the item is first worn, from the electronic device to the memory.

According to other advantageous characteristics of the method according to the invention, taken alone or in combination:
The activation operation is performed by manually scanning the memory with the electronic device.
The activation operation is performed by automatically detecting the memory with the electronic device, then carrying out a manual validation on the electronic device.
During the activation operation, the date of first wearing is recorded in a memory that is different from the memory of the item. For example, the date of first wearing is recorded in the electronic device and/or on the remote server.
The method comprises a notification operation consisting of warning the wearer when a predetermined period of time has elapsed since the item was first worn.

### BRIEF DESCRIPTION OF DRAWINGS

The invention will be better understood upon reading the following description, given solely as a non-limiting example, and made with reference to the accompanying figures wherein:

FIG. 1 is a schematic representation of a system for managing the wearing of an item, according to the invention; and

FIG. 2 is a perspective view of a building equipped with a system for managing the wearing of an item, according to a second embodiment of the invention.

### DETAILED DESCRIPTION

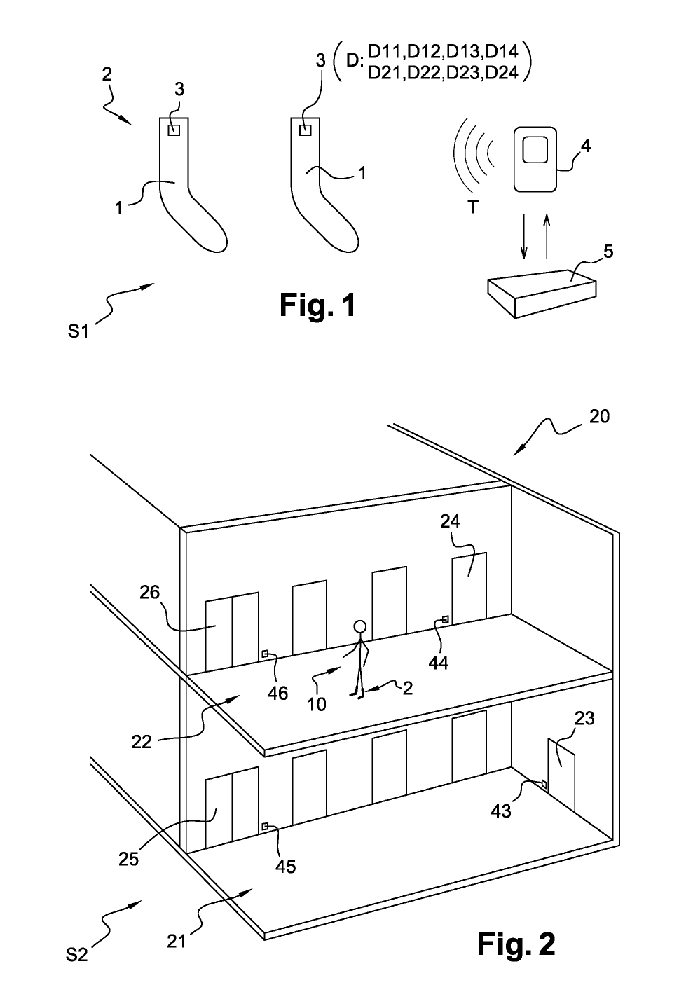

A system S1 for managing the wearing of the therapeutic items 1, in this instance compression socks, is represented in FIG. 1.

As an alternative, the items 1 can be any type of compression and/or contention items, designed to be fitted to the upper or lower limbs of a person.

The items 1 are designed to produce a therapeutic effect, for example the treatment of venous insufficiency. The items 1 constitute a pair 2 and are intended to be worn simultaneously.

Each item 1 incorporates a memory 3 for storing data D, for example in the form of an electronic chip. According to a particular example, the memory 3 can be an RFID (Radio Frequency Identification) chip, coupled to an RFID antenna.

According to another example, the memory 3 can be an NFC (Near Field Communication) chip, coupled to an NFC antenna. According to another example, the memory 3 may have no antenna.

The memory 3 comprises data D specific to the item 1 and/or the pair 2. For example, these data D can include an item reference D11, a pair identifier D12, an item type D13 and a date of manufacture D14. Preferably the data D11-D14 are recorded in the memory 3 during manufacture of the item 1. The data D11-D14 are fixed and unchangeable.

The memory 3 also comprises data D specific to the use of the item 1 and/or the pair 2. For example, the said data D can include an identifier of the wearer D21, a site identifier D22, an owner identifier D23 and a date D24 of first wearing. The data D21-D24 are not necessarily known during the manufacture of the item 1 and can be recorded in the memory 3 at a later stage. Once recorded in the memory 3, the data D21 and D24 are fixed and unchangeable during the life of the item 1.

The system S1 further comprises an electronic device 4 that is different from the item 1. The device 4 is configured to read the memory 3 of the item 1, more specifically the data D. The device 4 is further configured to write certain data D in the memory 3, in this instance the data D21-D24. Indeed, a transfer of data T is performed from the device 4 to the memory 3.

In practice, the identifier of the wearer D21 is recorded in the memory 3 when the pair 2 of items 1 is assigned to a particular wearer. The site identifier D22 is recorded in the memory 3 when the pair 2 is assigned to a particular site, such as a hospital or a nursing home. The identifier of the owner D23 is recorded in the memory 3 when the pair 2 is sold to a particular owner, for example the wearer or the site manager. The date D24 of first wearing is recorded in the memory 3 when the pair 2 is worn for the first time, or just prior to being worn for the first time, as detailed hereinafter.

The device 4 can be of any type suitable for the planned application, so that the transfer of data T can take place without a wired connection between the memory 3 and the device 4. In other words, the transfer T is performed by implementing a wireless communication standard.

The device 4 can be an RFID or NFC reader. The device 4 can be a smartphone, a digital tablet or a computer.

The device 4 preferably has an internal memory for storing data D related to each of the items 1. This is particularly useful when the system S1 comprises a plurality of pairs 2 of items 1 under management.

Optionally, the system S1 can comprise a remote server 5 for storing data D. For example, the device 4 can transmit the data D read and written in the memory 3 of each item 1 to the server 5. This is useful when the system S1 comprises a large number of pairs 2 of items 1 under management and/or when device 4 does not have sufficient internal memory.

Within the scope of the invention, an activation operation is performed for each item 1 of the pair 2 on the date the item 1 is first worn. This operation can be performed by the wearer or by another person. This operation is performed intentionally the day of the first wearing of the pairs 2 of items 1, with at least one manual intervention.

According to a preferred embodiment, the activation operation is performed by scanning the memory 3 of each item 1 with a device 4. In this case, the device 4 is preferably an NFC reader. The manual intervention consists of bringing the device 4 to within detection distance of the memory 3. Preferably, the scan is sufficient to perform the activation operation. As an alternative, a manual validation on the device 4 may be required after the scan in order to confirm the activation operation.

According to an embodiment variant, the activation operation can comprise automatic detection of the memory 3 by the device 4, then manual validation on the device 4. In this case, the device 4 is preferably an RFID reader. The memory 3 is detected by the device 4 when the item 1 is located within the predefined detection area around the device 4. Manual validation on the device 4 is required to confirm the activation operation. Manual validation makes it possible to prevent any accidental recording of data D in the memory 3.

The activation operation comprises a transfer of data T, including the date D24 the item 1 is first worn, from the device 4 to the memory 3 of the said item 1. Thus, the date D24 is recorded in the memory 3 of each item 1. Preferably, the date D24 is also recorded in the device 4 and/or on the server 5. The date D24 of first wearing comes from the device 4, and not directly from the item 1.

Afterwards, the wearer of the item 1 can be informed of the period of time elapsed since the date D24. This information can be obtained at any time, for example by scanning the memory 3 with the device 4. As an alternative, this information can be obtained automatically, for example in the form of a notice sent to the wearer and/or to another person by the device 4 or the server 5. Advantageously, said notice can be sent a short time before the item 1 reaches end of life and/or when the item 1 reaches end of life.

For example, the working life of the pair 2 of items 1 can be fixed at six months, after which said item 1 no longer provides sufficient therapeutic effect. The wearer must then change pair 2 in order to benefit from the desired therapeutic effect.

According to a particular embodiment, the item 1 may be provided with a sensor for detecting the wearing by the wearer, for example in the form of a conductive wire or pressure switch. This makes it possible to know the times of wearing of the item 1 by the wearer, and thus to have additional information about compliance.

As an alternative, the item 1 may not have a sensor for detecting wearing by the wearer. The item 1 is then simpler and less expensive to manufacture. The key information is the time elapsed from the date D24 of first wearing.

A second embodiment of a system S2 for managing the wearing of therapeutic items 1 according to the invention is represented in FIG. 2.

The system S2 is installed in a building 20. A wearer 10, equipped with a pair 2 of items 1, lives and moves about within the building 20. For example, the building 20 is a nursing home, the wearer 10 is an elderly person, and the items 1 are socks.

The system S2 includes the electronic device 4 of FIG. 1, not shown in FIG. 2 for purposes of simplification. An activation operation is performed with the device 4 when the wearer 10 puts on the pair 2 for the first time, so that the date D24 of first wearing of each item 1 is recorded in the memory 3.

The building 20 comprises a first floor 21 and a second floor 22, each having several doors 23, 24, 25, 26 and several rooms. The first floor 21 includes an entry door 23 and an elevator door 25. The second floor 22 includes a door 24 to a room and an elevator door 26. The door 24 is the one to the room of the wearer 10.

The system S2 comprises various electronic devices 43, 44, 45 and 46, positioned respectively beside the doors 23, 24, 25 and 26. The devices 43-46 are configured to read the data D recorded in the memory 3 of the items 1 when the wearer 10 passes nearby. The devices 43-46 are for example RFID or NFC sensors.

The devices 43-46 can be connected to the device 4 and/or to the remote server 5 of FIG. 1, in order to transmit thereto the data D read in the memory 3. Each read operation can be associated with a device identifier 43-46 and a read time (date and time). Thus, the network of devices 43-46 makes it possible to track the movements of the wearer 10 in the building.

In addition or as an alternative, the memory 3 can be configured to store the device identifier 43-46 and the read time corresponding to each read operation.

The system S2 makes it possible to track the movements of the wearer 10 in the building when the wearer 10 is wearing the items 1. The personnel of the building 20 can thus know the approximate position of the wearer 10 in the building 20. According to one particular embodiment, a visual or audible alert can be issued when the wearer 10 enters an unauthorized area, for example when the wearer 10 approaches too close to the entry door 23.

The system S2 further makes it possible to ensure that the wearer 10 is properly wearing the items 1 belonging to the same pair 2 and having the same date D24. This is particularly important in terms of compliance.

When there is a large number of wearers 10 and items 1 are present in the building 20, the data D11-D14 and D21-D24 facilitate the management by the site operator of the items 1.

Irrespective of the embodiment of the invention, the memory 3 of each item 1 is configured to record the date D24 said item 1 is first worn, in an activation operation performed the day the item is first worn 1 and which comprises a transfer of data T (including the date D24) from the electronic device 4 to the memory 3.

Furthermore, the items 1 and the management systems S1/S2 can be configured differently from FIGS. 1 to 2 without going beyond the scope of the invention. Moreover, the technical characteristics of the various embodiments and variants mentioned above can be, in whole or for some of them, combined with each other. Thus, the items 1 and the systems S1/S2 may be adapted in terms of cost, functionality and performance.

1. Compression and/or contention item designed to be fitted to a limb of a wearer, the item incorporating a memory for storing data, which can be read, and for some written, by a distinct electronic device, the memory including at least one datum for identifying the item, wherein the memory is configured to record a date on which the item is first worn, in an activation operation that is carried out on the day the item is first worn and which comprises a transfer of data, including the date on which the item is first worn, from the electronic device to the memory.

2. The item according to claim 1, wherein the item is devoid of a sensor for detection of wearing by the wearer.

3. The item according to claim 1, wherein the item is devoid of a battery.

4. The item according to claim 1, wherein the memory includes at least one datum of identification of the item chosen among the following:
an item reference;
a pair identifier, when two items must be worn simultaneously;
an item type; and/or
a date of manufacture.

5. The item according to claim 1, wherein the at least one datum of identification of the item is fixed and unchangeable in the memory.

6. The item according to claim 1, wherein the memory includes a wearer identifier, a site identifier and/or an owner identifier.

7. The item according to claim 1, wherein the item is equipped with RFID or NFC communication means for communication with the electronic device.

8. The item according to claim 7, wherein the communication means are activated only in the event the data are read or written by the electronic device in the memory.

9. Pair composed of two items according to claim 1.

10. A system for managing the wearing of an item, comprising at least:
an item according to claim 1; and
an electronic device configured to read the data and write at least some of the data in the memory incorporated in the item.

11. Method of managing the wearing of a compression and/or contention item designed to be fitted to a limb of a wearer, the item incorporating a memory for storing data, which can be read and for some written by a distinct electronic device, the memory including at least one datum for identifying the item,
wherein the method comprises an activation operation performed on the day the item is first worn and which comprises a transfer of data, including the date on which the item is first worn, from the electronic device to the memory.

12. The method according to claim 11, wherein the activation operation is performed by manually scanning the memory with the electronic device.

13. The method according to claim 11, wherein the activation operation is performed by automatically detecting the memory with the electronic device, then carrying out a manual validation on the electronic device.

14. The method according to claim 11, wherein during the activation operation, the date of first wearing is recorded in a second memory different from the memory of the item.

15. The method according to claim 11, wherein the method comprises a notification operation consisting of warning the wearer when a predetermined period of time has elapsed since the item was first worn.

16. The method according to claim 14, wherein during the activation operation, the date of first wearing is recorded in the electronic device.

17. The method according to claim 14, wherein during the activation operation, the date of first wearing is recorded on a remote server.
